(19) United States
(12) Patent Application Publication
(10) Pub. No.: US 2024/0382390 A1
(43) Pub. Date: Nov. 21, 2024

(54) LIQUID CRYSTAL COSMETIC COMPOSITION WITH EXCELLENT ELECTRICAL CONDUCTIVITY FOR FORMING MICROCURRENT

(71) Applicant: NEW&NEW Co., Ltd., Cheonan-si (KR)

(72) Inventors: Hyo Tae KIM, Seoul (KR); Gu Hyun KANG, Seongnam-si (KR); Hyung Loul KIM, Cheonan-si (KR); Ho Young LEE, Asan-si (KR)

(73) Assignee: NEW&NEW Co., Ltd., Cheonan-si (KR)

(21) Appl. No.: 18/376,238

(22) Filed: Oct. 3, 2023

(30) Foreign Application Priority Data
May 17, 2023 (KR) 10-2023-0063984

(51) Int. Cl. A61K 8/02; A61Q 19/00

(52) U.S. Cl. CPC A61K 8/0295; A61Q 19/00; A61K 2800/26; A61K 2800/48; A61K 2800/524; A61K 2800/83

(57) ABSTRACT

Provided is a liquid crystal cosmetic composition with excellent electrical conductivity for forming microcurrents. The liquid crystal cosmetic composition is capable of increasing electrical conductivity, forming a voltage, acting as a chemical battery, and forming a microcurrent, based on an appropriate combination of a liquid crystal emulsion with an electrolyte, thereby being highly effective in increasing absorption on the skin through ionization of active ingredients, and in improving skin elasticity and strengthening the skin barrier through the microcurrent.

### CROSS REFERENCE TO RELATED APPLICATIONS

This application claims priority based on Korean Patent Application No. 10-2023-0063984 filed May 17, 2023, the entire disclosures of which is incorporated herein by reference.

### BACKGROUND OF THE INVENTION

### Field of the Invention

The present invention relates to a liquid crystal cosmetic composition with excellent electrical conductivity for forming microcurrents and more specifically, to a liquid crystal cosmetic composition that increases electrical conductivity and generates a voltage to form microcurrent through a combination of a liquid crystal emulsion with an electrolyte.

### Description of the Related Art

Many years of research showing that microcurrents have beneficial functions such as wound healing, cellular activity, skin tone improvement, and skin elasticity have recently been published. Therefore, the beauty device market using microcurrents has continued to grow steadily in recent years. However, beauty devices have the disadvantages of high price and inconvenience to use and carry. Accordingly, there is a need for development of a cosmetic formulation that can generate microcurrents without a beauty device.

Accordingly, recently, the concept of piezoelectricity (a phenomenon in which a microcurrent or voltage is generated by polarization when force is applied) has been applied to cosmetics based on the method by which consumers use cosmetics by applying the same to the skin, followed by tapping or rubbing. In other words, a microcurrent generation mechanism in which a microcurrent is formed when pressure is applied during application has been designed.

However, there is a need for a novel method for generating microcurrents without direct application of a pressure, effectively delivering active ingredients to the skin layer, increasing absorption thereof on the skin, and providing beneficial functionality to the skin (enhancing elasticity, strengthening the skin barrier, and the like) based on microcurrents.

### PRIOR ART

### Patent Document

(Patent Document 1) Korea Patent No. 10-2041748 (registered on Oct. 31, 2019) relates to a cosmetic composition having piezoelectric performance, and more specifically, to a cosmetic composition having piezoelectric performance that contains an emulsifier, a polymer powder, an emulsion stabilizer, a piezoelectric polymer, and water, and that can thus effectively deliver water-soluble active ingredients to the skin without any cosmetic device.

(Patent Document 2) Korea Patent No. 10-1819467 (registered on Jan. 11, 2018) relates to a piezoelectric cosmetic composition, and more specifically, relates to a piezoelectric cosmetic composition containing, as an active ingredient, a piezoelectric material that generates a microcurrent when pressure is applied thereto.

### SUMMARY OF THE INVENTION

It is highly required to apply microcurrents to cosmetic compositions because microcurrents can exert beneficial functions on the skin. However, conventional devices that are expensive and inconvenient to use and carry had limited applications. Accordingly, it is an object of the present invention to provide a liquid crystal cosmetic composition that is capable of increasing electrical conductivity, forming a voltage, acting as a chemical battery, and forming a microcurrent, based on an appropriate combination of a liquid crystal emulsion with an electrolyte, thereby providing potent effects of increasing absorption through the skin, of improving skin elasticity, and of strengthening the skin barrier.

In accordance with the present invention, the above and other objects can be accomplished by the provision of a method for preparing a liquid crystal cosmetic composition, including (a) adding a thickener to an aqueous phase, followed by dispersion, (b) adding a well-dissolved oil phase to the aqueous phase of step (a), followed by emulsification, and (c) allowing the resulting emulsion to cool to form liquid crystal particles, wherein the aqueous phase contains purified water, glycerin, hydrogenated lecithin, cetearyl olivate, sodium citrate, citric acid, and betaine, the thickener contains diutan gum and xanthan gum, and the oil phase contains shea butter, cetearyl alcohol, stearic acid, phenylmethyl trimethicone, caprylic and capric triglyceride, and polyglyceryl-6 stearate.

Meanwhile, the liquid crystal cosmetic composition may generate microcurrents.

Meanwhile, the aqueous phase may further contain a preservative.

Meanwhile, the diutan gum may be derived from a culture medium of a Sphingomonas sp. strain containing diutan gum or an extract thereof.

In accordance with another aspect of the present invention, provided is a liquid crystal cosmetic composition prepared by the method.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is an image showing a liquid crystal cosmetic composition of the present invention observed with a microscope;

FIG. 2 shows a measuring tool used to measure the current and microcurrent of the liquid crystal cosmetic composition of the present invention;

FIG. 3 shows the measured electrical conductivity of the liquid crystal cosmetic composition of the present invention;

FIG. 4 shows the electrical conductivity measured on the skin to which the liquid crystal cosmetic composition of the present invention is applied; and

FIG. 5 is an image showing light emission of a light-emitting diode, to which the liquid crystal cosmetic composition of the present invention is applied, compared to a cosmetic composition from another company.

### DETAILED DESCRIPTION OF THE INVENTION

Hereinafter, the present invention will be described in detail.

It is highly required to apply microcurrents to cosmetic compositions because microcurrents can exert beneficial functions on the skin. However, conventional devices that are expensive and inconvenient to use and carry had limited applications. Accordingly, recently, a microcurrent generation method for generating microcurrents when pressure is applied to the skin, to which cosmetics are applied, has been designed based on the concept of piezoelectricity (a phenomenon in which a microcurrent or voltage is generated by polarization when force is applied) has been applied to cosmetics based on the method by which consumers use cosmetics by applying the same to the skin, followed by tapping or rubbing.

Meanwhile, the present invention aims at designing a effectively generate cosmetic composition that can microcurrent without applying such direct pressure. As a result of extensive efforts, the present inventors found that microcurrents can be generated within a formulation without applying pressure to the skin or performing application of active ingredients to the skin by increasing electrical conductivity, forming voltage and thus providing function as a chemical battery, based on fluidity and complementarity through an appropriate combination of a liquid crystal emulsion with an electrolyte.

Accordingly, the present invention provides a method for preparing a liquid crystal cosmetic composition, including: (a) adding a thickener to an aqueous phase, followed by dispersion; (b) adding a well-dissolved oil phase to the aqueous phase of step (a), followed by emulsification; and (c) allowing the resulting emulsion to cool to form liquid crystal particles, wherein the aqueous phase contains purified water, glycerin, hydrogenated lecithin, cetearyl olivate, sodium citrate, citric acid, and betaine, the thickener contains diutan gum and xanthan gum, the oil phase contains shea butter, cetearyl alcohol, stearic acid, phenylmethyl trimethicone, caprylic and capric triglyceride, and polyglyceryl-6 stearate.

The liquid crystal cosmetic composition prepared by the method described above can generate microcurrents well due to excellent electrical conductivity thereof, and has an effect of improving skin absorption through ionization of the active substances in the cosmetic composition, called “galvanic effect”, and effects of improving skin elasticity and strengthening the skin barrier based on the microcurrent.

The method of preparing the liquid crystal cosmetic composition of the present invention will be described in detail in each step as follows.

### Step (a): Adding Thickener to Aqueous Phase, Following by Dispersion

In this step, a thickener is added to an aqueous phase, followed by dispersion. The aqueous phase contains purified water, glycerin, hydrogenated lecithin, cetearyl olivate, sodium citrate, citric acid, and betaine.

Meanwhile, in this step, the aqueous phase is heated to 60 to 80° C., and the thickener is added to and dispersed in the aqueous phase, followed by heating to 70-90° C. Here, heating to a temperature below the lower limit of the range may affect the solubility of the oil phase or have a negative effect on particle size formation, and heating to a temperature above the upper limit of the range may have a negative effect on emulsion formation due to high amounts of evaporation.

Hydrogenated lecithin is an ingredient obtained by adding hydrogen to lecithin derived from beans or eggs. Hydrogenated lecithin is prepared by adding hydrogen to lecithin because lecithin becomes unstable when it comes in contact with oxygen. Hydrogenated lecithin is known to be used as a surfactant (emulsifier), a suspending agent (non-surfactant), a skin conditioner, or the like, and can exert moisturizing, elasticizing and skin-recovering effects. Here, hydrogenated lecithin was used as a key ingredient to prepare a liquid crystal emulsion.

Cetearyl olivate is a synthetic ester made with fatty acids derived from olive oil, which is combined with cetearyl alcohol, and is mainly present in a liquid phase at room temperature. It is used as a vegetable emulsifier ingredient that can replace a PEG-based emulsifier or PPG-based emulsifier, and can be used as an ingredient with various effects such as moisturizing, skin barrier recovering, and skin soothing effects. In the present invention, cetearyl olivate was used as a key ingredient to prepare a liquid crystal emulsion.

Meanwhile, in the present invention, a microcurrent can be generated within the formulation itself by combining a liquid crystal emulsion with an electrolyte. To this end, sodium citrate, citric acid, and betaine are used as electrolytes. At this time, a liquid crystal emulsion is formed in the form of multiple layers and ingredients of the electrolyte are disposed between the layers, to increase electrical conductivity and form a microcurrent.

Sodium citrate refers to a sodium salt of citric acid and is in the form of a salty white powder. Sodium citrate is used as a metal ion sequestrant to prevent cosmetics from spoiling or deteriorating, and as a pH (acidity) regulator to control the acidity of cosmetics, preserve the color thereof, and prevent oxidation thereof. In the present invention, it was used as one of electrolyte ingredients.

Citric acid is a type of organic acid, also called “fruit acid” and is a natural ingredient extracted from sour fruits. It is used as a metal ion sequestrant to prevent cosmetics from spoiling or deteriorating, a fragrance, a pH regulator to prevent oxidation thereof. In the present invention, it was used as one of electrolyte ingredients.

Betaine is an amino acid extracted from sugar beets, beets, Lycium chinense (goji berries) and the like, and acts as a moisturizer, a hair conditioner, a food additive, an antioxidant and the like. This exhibits an effect of preventing skin drying by controlling the balance between oil and moisture, an anti-inflammatory effect, and an effect of reducing irritation from cosmetic ingredients. In the present invention, it was used as one of the electrolytes.

Meanwhile, the electrolyte preferably contains 0.04 to 0.06 parts by weight of sodium citrate, 0.04 to 0.06 parts by weight of citric acid, and 1 to 3 parts by weight of betaine. As a result, the electrolyte acts well as a chemical battery through the stable synergistic effect with the liquid crystal emulsion.

Meanwhile, in the preparation method of the present invention, the aqueous phase preferably further contains a preservative. Any preservative may be used as the preservative without limitation so long as it is well-known in the art.

Meanwhile, in this step, any thickener may be used as the thickener without limitation so long as it is well-known in the art. Examples of the thickener include natural substances such as casein, alginic acid, and xanthan gum, semi-synthetic substances such as carboxymethylcellulose and methylcellulose, and synthetic polymers such as carboxy vinyl polymers, and polyacrylates. Preferably, the thickener contains at least one selected from diutan gum and xanthan gum. The diutan gum is preferably culture media of Sphingomonas sp. strains containing diutan gum or diutan gum derived from extracts thereof. The purified culture medium or extract thereof was used because, when Sphingomonas sp. strains containing diutan gum are cultured, a large amount of diutan gum is secreted into the culture medium. More preferably, the thickener contains 0.1 to 0.3 parts by weight of diutan gum and 0.02 to 0.06 parts by weight of xanthan gum.

### Step (b): Adding Well-Dissolved Oil Phase to Aqueous Phase of Step (a), Followed by Emulsification

In step (b), the well-dissolved oil phase is added to the aqueous phase of step (a), followed by emulsification. The oil phase contains shea butter, cetearyl alcohol, stearic acid, phenylmethyl trimethicone, caprylic and capric triglyceride, and polyglyceryl-6 stearate.

Meanwhile, in this step, preferably, the oil phase is heated to 70-90° C., dissolved well, added to the aqueous phase of step (a) and emulsified. This enables effective emulsification of the oil phase and the aqueous phase. Here, heating to a temperature below the lower limit of the range may prevent the oil phase from being dissolved and heating to a temperature above the upper limit of the range may cause problems associated with rancidity of fatty acids.

Meanwhile, in the present invention, in order to prepare a liquid crystal emulsion, any fatty acid and emulsifier known in the art may be used. Preferably, a liquid crystal emulsion can be well formed by emulsifying hydrogenated lecithin and cetearyl olivate of the aqueous phase, and stearyl alcohol and stearic acid of the oil phase. Surfactants such as hydrogenated lecithin, cetearyl olivate, stearic acid, and cetearyl alcohol form emulsified particles as multiple layers, thus enabling the stable formation of a liquid crystal emulsion. In this case, the surfactants, during emulsification, form double layers which are granulated to form a multilayer liquid crystal emulsion. In addition, cetearyl alcohol is a solid at room temperature and thus can retain the shape thereof stably when cooled, thus enabling effective formation liquid of a crystal emulsion. Preferably, the liquid crystal emulsion contains 0.4 to 0.6 parts by weight of hydrogenated lecithin, 1 to 2 parts by weight of cetearyl olivate, 1 to 3 parts by weight of cetearyl alcohol, and 1 to 3 parts by weight of stearic acid.

Cetearyl alcohol is a mixture of fatty alcohols composed of cetyl and stearyl alcohol, and is widely used as an emulsion stabilizer, an opacifying agent, a surfactant-foaming agent, or a thickener. This is effective in improving the applicability of cosmetics and moisturizing and thus can be used as an ingredient for various cosmetics. In the present invention, it was used as one of the key ingredients to prepare a liquid crystal emulsion.

Stearic acid is a mixture of higher saturated fatty acids derived from natural fatty acids, which is also called “octadecanoic acid”. It is used as a fragrance, surfactant, detergent, or emulsifier, and is involved in the stabilization of immiscible different ingredients, when adsorbed to the interface between the substances, to provide cleaning power. In addition, it has excellent skin stability and forms an oil film on the skin, thus preventing moisture from evaporating and preventing skin dryness. In the present invention, it was used as one of the key ingredients to prepare a liquid crystal emulsion.

### Step (c): Allowing Resulting Emulsion to Cool to Form Liquid Crystal Particles

In this step, the product obtained in step (b) is allowed to cool to form liquid crystal particles.

Meanwhile, in this step, after emulsification, cooling is preferably performed to 30-50° C. to form liquid crystal particles. Here, disadvantageously, cooling to the temperature out of the range may cause the particles to be deformed due to insufficient cooling.

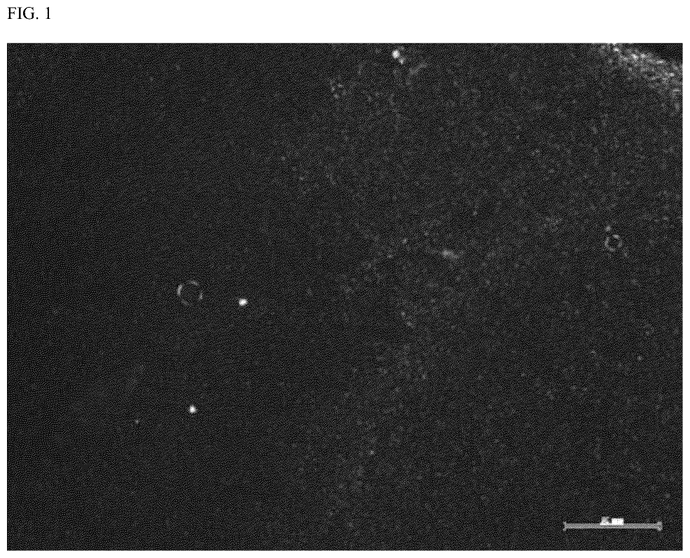

As such, the liquid crystal cosmetic composition is completed using the method described above (FIG. 1). The liquid crystal cosmetic composition is capable of increasing electrical conductivity, forming a voltage, acting as a chemical battery and forming a microcurrent based on an appropriate combination of the liquid crystal emulsion and the electrolyte, thereby providing potent effects of increasing absorption through the skin, of improving skin elasticity, and of strengthening the skin barrier.

Meanwhile, in the liquid crystal cosmetic composition of the present invention, the cosmetic composition may be utilized in at least one application selected from the group consisting of increasing skin absorption, improving skin elasticity, and strengthening the skin barrier. In addition, formation of microcurrents provides the effects of increasing skin absorption through ionization of the active ingredients, improving skin elasticity and strengthening the skin barrier.

Meanwhile, the cosmetic composition of the present invention may be selected from: skin care formulations including solutions, suspensions, emulsions, pastes, skin toner, gels, water-soluble liquids, creams, essences, surfactant-containing cleansing, oils, oil-in-water (O/W) and water-in-oil (W/O) emulsions, lotions, eye creams, soothing gels, and ointments; formulations for mask packs; formulations for body wash; peeling gels; color makeup formulations including oil-in-water (O/W) and water-in-oil (W/O) makeup bases, foundations, skin covers, lipsticks, lip glosses, face powders, two-way cakes, eye shadows, cheek colors and eyebrow pencils; and formulations for scalp. Preferably, the cosmetic composition includes at least one formulation selected from the group consisting of transparent skin toner, transparent essence, low-viscosity emulsified lotion essences, and high-viscosity emulsified cream.

In addition, the cosmetic composition of the present invention may contain one or more additives ordinarily used in the field of cosmetics, for example, hydrophilic or lipophilic activators, preservatives, antioxidants, solvents, air fresheners, fillers, blockers, pigments, odor-absorbers, dyes and the like. The amounts of these various additives are those commonly used in the art. In any case, the additives and proportions thereof will be selected so as not to adversely affect the desirable properties of the cosmetic composition according to the present invention.

Furthermore, the cosmetic composition of the present invention may be used in combination with another cosmetic composition. Further, the cosmetic composition according to the present invention may be used in accordance with a conventional use method and the number of uses may be varied depending on the skin condition or preference of the user.

Meanwhile, it can be seen from the following experiment that the liquid crystal cosmetic composition of the present invention prepared by mixing the liquid crystal emulsion with the electrolyte has excellent effects of forming a current and voltage, improving electrical conductivity, and generating high luminescence. In reality, even when applied to the skin, microcurrents could be well formed. This indicates that the liquid crystal cosmetic composition of the present invention forms a microcurrent, thereby providing an effect of improving skin absorption through ionization of the active substances in the cosmetic composition, called “galvanic effect”, and effects of improving skin elasticity and strengthening the skin barrier through the microcurrent, thereby being applicable as a variety of cosmetic ingredients. Hereinafter, the present invention will be described in more detail with reference to the following examples and experimental examples, but the scope of the present invention is not limited to the examples and experimental examples, and includes variations and technical concepts equivalent thereto.

### EXAMPLE AND COMPARATIVE EXAMPLE: PREPARATION OF LIQUID CRYSTAL COSMETIC COMPOSITION OF PRESENT INVENTION FOR FORMING MICROCURRENTS

In this example, a liquid crystal cosmetic composition that forms microcurrents was prepared. For comparison, a cosmetic composition not containing an electrolyte was prepared. The details of the composition are shown in Table 1 below. Hydrogenated lecithin, cetearyl olivate, cetearyl alcohol, and stearic acid were used to form a liquid crystal emulsion, and sodium citrate, citric acid, and betaine were added as electrolytes.

The process for preparing the liquid crystal cosmetic composition of the present invention is as follows. First, an aqueous phase (containing ingredients 1 to 8 in Table 1) was heated to 70° C., and a thickener (ingredients 9 to 10 in Table 1) was well dispersed therein, following by heating to 80° C. The oil phase (containing ingredients 11 to 16 in Table 1) was heated to 80° C., dissolved well, and then added to the aqueous phase for emulsification. Then, the resulting product was slowly cooled to 40° C. to form liquid crystal particles well. The liquid crystal cosmetic composition obtained in this way was observed with a microscope, as shown in FIG. 1, and an experiment was performed below to determine whether or not a microcurrent was formed well.

TABLE 1

| | | Content (%) | |
|---|---|---|---|
| | | | Comparative |
| Ingredient | | Example | Example |
| Aqueous phase | 1. Purified water | to 100 | to 100 |
| | 2. Glycerin | 5 | 5 |
| | 3. Preservative | 2 | 2 |
| | 4. Hydrogenated lecithin | 0.5 | 0.5 |
| | 5. Cetearyl olivate | 1.5 | 1.5 |
| | 6. Sodium citrate | 0.05 | 0 |
| | 7. Citric acid | 0.05 | 0 |
| | 8. Betaine | 2 | 0 |
| Thickening | 9. Diutan gum * | 0.15 | 0.15 |
| agent | 10. Xanthan gum | 0.04 | 0.04 |
| Oil phase | 11. Shea butter | 5 | 5 |
| | 12. Cetearyl alcohol | 2 | 2 |
| | 13. Stearic acid | 2 | 2 |
| | 14. Phenylmethyl | 1 | 1 |
| | trimethicone | | |
| | 15. Caprylic and capric | 14 | 14 |
| | triglyceride | | |
| | 16. Polyglycerol-6 | 1 | 1 |
| | stearate | | |

* Diutan gum was derived from the culture medium of a Sphingomonas sp. strain containing diutan gum.

### Experimental Example 1: Measurement of Voltage, Microcurrent, and Electrical Conductivity of Liquid Crystal Cosmetic Composition of Present Invention, and Confirmation of Light Emission level Thereof

In this experimental example, the voltage, microcurrent, and electrical conductivity of the liquid crystal cosmetic composition of Example above were measured, and the degree of light emission of a light emitting diode, to which the liquid crystal cosmetic composition was applied, was determined.

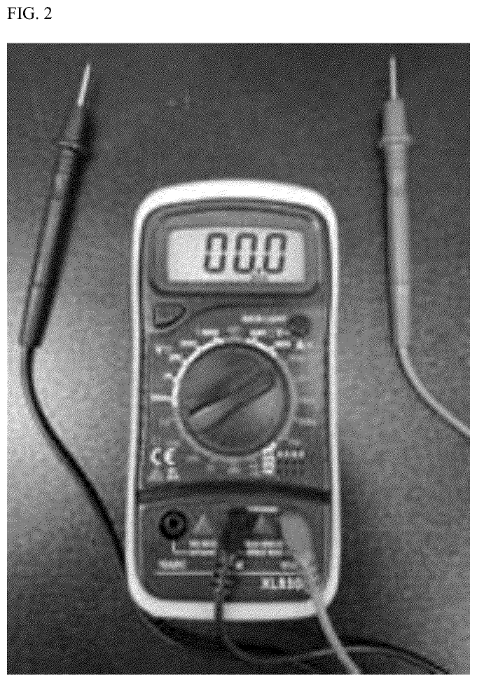

First, voltages and microcurrents were measured using the measurement tool shown in FIG. 2. As shown in Table 2, the result showed that the liquid crystal cosmetic composition of the present invention prepared by combining the liquid crystal emulsion with electrolyte generated a considerably high current and voltage, compared to Comparative Example. In addition, it was found that the liquid crystal cosmetic composition exhibited much higher current and voltage formation, compared to a galvanic cream from other company.

TABLE 2

| | | Comparative | Control group (galvanic |
|---|---|---|---|
| | Example | Example | cream from other company) |
| Current (nA) | 38.94 | 10 | 2 |
| Voltage (μV) | 8461.55 | 2425.15 | 1350.25 |

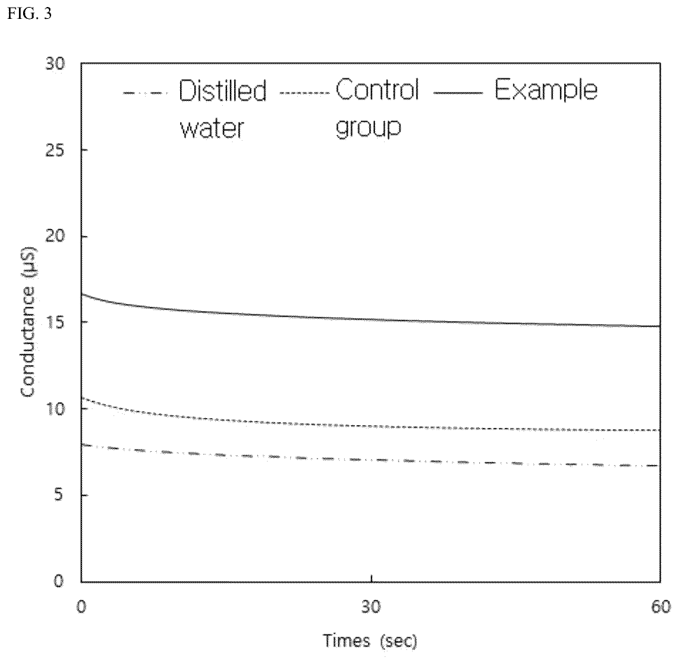

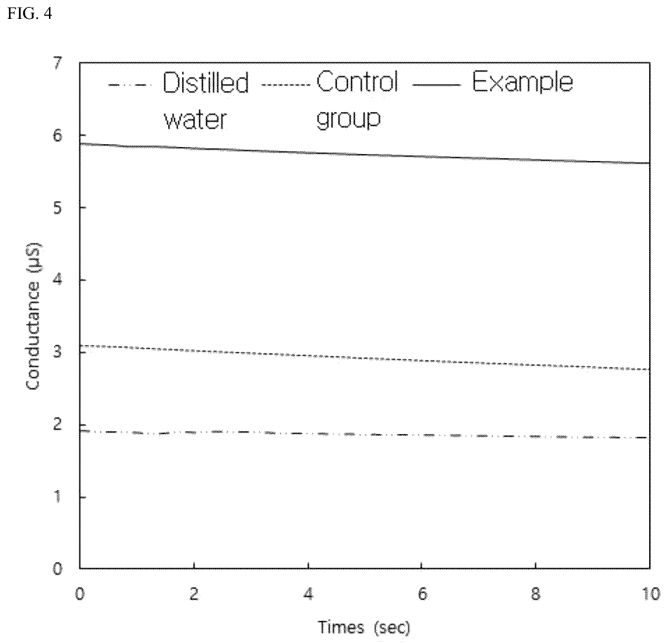

Accordingly, the electrical conductivity of the liquid crystal cosmetic composition of the present invention was measured. As shown in FIG. 3, the result showed that the liquid crystal cosmetic composition exhibited excellent electrical conductivity compared to a control group (galvanic cream from another company) and distilled water, which indicates that microcurrent was well formed. In addition, as shown in FIG. 4, the electrical conductivity measured on the skin, to which the liquid crystal cosmetic composition was applied, was superior to that of the control group (galvanic cream from another company) and distilled water, which indicates that the liquid crystal cosmetic composition produced microcurrents well.

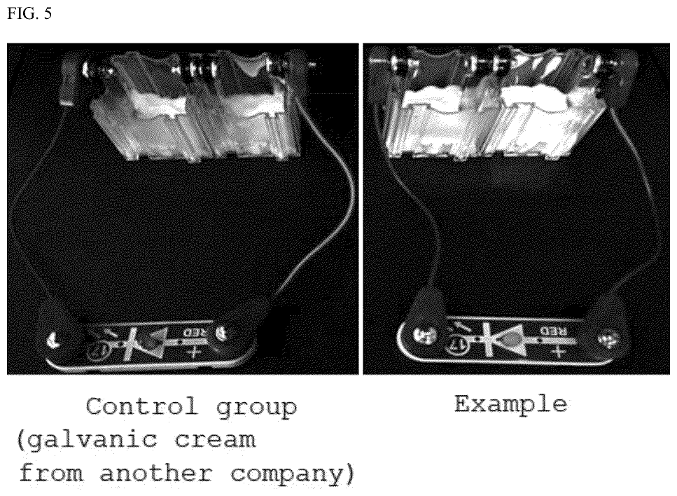

Meanwhile, the degree of light emission measured on the light emitting diode, to which the liquid crystal cosmetic composition of the present invention was applied, was excellent, compared to Control Group (galvanic cream from another company), as shown in FIG. 5.

Overall, it was found that the liquid crystal cosmetic composition of the present invention exhibits higher current voltage, and electrical conductivity, and induces superior light emission. This indicates that the liquid crystal cosmetic composition of the present invention forms a microcurrent, when applied to the skin, thus providing an effect of improving skin absorption through ionization of the active substances in the cosmetic composition, called “galvanic effect”, and effects of improving skin elasticity and strengthening the skin barrier through the microcurrent.

As apparent from the foregoing, the present invention provides a liquid crystal cosmetic composition that is capable of increasing electrical conductivity, forming a voltage, acting as a chemical battery, and forming a microcurrent, based on an appropriate combination of a liquid crystal emulsion with an electrolyte, thereby providing potent effects of increasing absorption on the skin through ionization of active ingredients, of improving skin elasticity, and of strengthening the skin barrier through the microcurrent.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method for preparing a liquid crystal cosmetic composition, comprising:
(a) adding a thickener to an aqueous phase, followed by dispersion;
(b) adding a well-dissolved oil phase to the aqueous phase of step (a), followed by emulsification; and
(c) allowing the resulting emulsion to cool to form liquid crystal particles,
wherein the aqueous phase comprises purified water, glycerin, hydrogenated lecithin, cetearyl olivate, sodium citrate, citric acid, and betaine,
the thickener comprises diutan gum and xanthan gum, and
the oil phase comprises shea butter, cetearyl alcohol, stearic acid, phenylmethyl trimethicone, caprylic and capric triglyceride, and polyglyceryl-6 stearate.

2. The method according to claim 1, wherein the liquid crystal cosmetic composition generates microcurrents.

3. The method according to claim 1, wherein the aqueous phase further comprises a preservative.

4. The method according to claim 1, wherein the diutan gum is derived from a culture medium of a Sphingomonas sp. strain containing diutan gum or an extract thereof.

5. A liquid crystal cosmetic composition prepared by the method according to claim 1.

6. A liquid crystal cosmetic composition prepared by the method according to claim 2.

7. A liquid crystal cosmetic composition prepared by the method according to claim 3.

8. A liquid crystal cosmetic composition prepared by the method according to claim 4.
